(19) United States
(12) Patent Application Publication
(10) Pub. No.: US 2002/0128676 A1
(43) Pub. Date: Sep. 12, 2002

(54) Finger pressure device for acupuncture

(76) Inventor: Tae Woo Yoo, Seoul (KR)

Correspondence Address: Richard M. Goldberg, Suite 419, 25 East Salem Street, Hackensack, NJ, 07601, US

(21) Appl. No.: 10/016,387

(22) Filed: Dec. 10, 2001

(30) Foreign Application Priority Data
Mar. 7, 2001 (KR) 2001-6029

(51) Int. Cl. A61B 17/34

(52) U.S. Cl. 606/189

(57) ABSTRACT

A finger pressure device for acupuncture comprising: an adhesive tape with an adhesive surface on one side; a T-pellet attached on the adhesive surface of said adhesive tape; an one-sided tape with a small hole in the center, having an adhesive surface of the same size with said adhesive tape; and a gold plate with a small hole in the center through which an end portion of said T-pellet is inserted.

### BACKGROUND OF THE INVENTION

1. Field of the invention

The present invention relates to a finger pressure device for acupuncture, and more particularly, to a finger pressure device for acupuncture for providing an enhanced pressure stimulation effect by the T-pellet, a scattering effect of excessive body current and promoting a fluent body current circulation effect.

A human heart creates a body current of 10 micro ampere and transmits into the whole body. If the body current is transmitted into all the organs in a good balance, the body feels good and has no disease. But in case that excessive currents are transmitted into some parts, the body might get fever, pain, inflammation and so on. Conversely, in case that some parts get short of required currents, the body might experience psychroalgia, malfunction, paralysis, blood circulation disorder, trembling, spasm and so on.

2. Description of the Prior Art

In general, electronic needles were used in acupuncture therapy in order to control the imbalance of body current as above. The electronic needles were helpful to control the imbalance of body currents, but sometimes cause dizziness, fatigue, fever, paralysis, hallucination, powerlessness, since these electronic needles create pretty strong currents of between minimum 100 micro ampere and maximum 800 micro ampere.

An alternative to the electronic needles was a gold string inserted into Meridian points in the skin or the affected parts so as to keep balance in transmitting body currents. This treatment could be also dangerous to the body due to lack of safety and was not economical since the string is made of expensive gold.

And another solution to the above problem was a finger pressure device which is the prior art of the present invention, including an adhesive tape with a small hole in the center to be inserted by an end portion of a T-pellet. But the T-pellet of this device provides just pressure-stimulation effects to the Meridian points of the skin or the affected parts, but does not offer any other treatment effects.

### SUMMARY OF THE INVENTION

The present device was designed to overcome the disadvantages described as above. An object of the present invention is to provide a T-pellet for acupuncture which has enhanced pressure stimulation effects due to the end portion of T-pellet.

Another object of the present invention is to provide a T-pellet for acupuncture which has a scattering effect of excessive body current in some parts of the body, since the gold plate of the T-pellet for acupuncture has a great electric conduction. In case that excessive currents are transmitted into some parts, the body might get fever, pain, inflammation and so on.

The third object of the present invention is to provide a T-pellet for acupuncture which promotes a fluent body current conduction in some parts of the body. In case that a person gets short of required body current, the body might experience psychroalgia, malfunction, paralysis, blood circulation disorder, trembling, spasm and so on.

### BRIEF DESCRIPTION OF THE DRAWINGS

The other objects and features of the present invention will be hereinafter explained in detail with reference to the accompanying drawings, wherein:

FIG. 1 is a disassembled perspective view of the present invention.

FIG. 2 is an assembled perspective view of the present invention.

FIG. 3 is an exploded cross-sectional view showing a state of a use of the present invention.

FIG. 4 is an exemplary view showing a state of a use of the present invention.

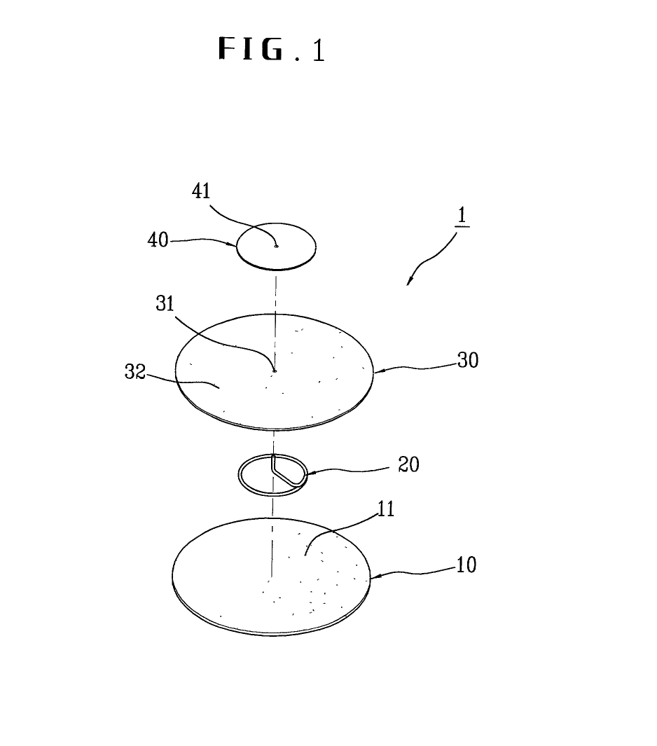

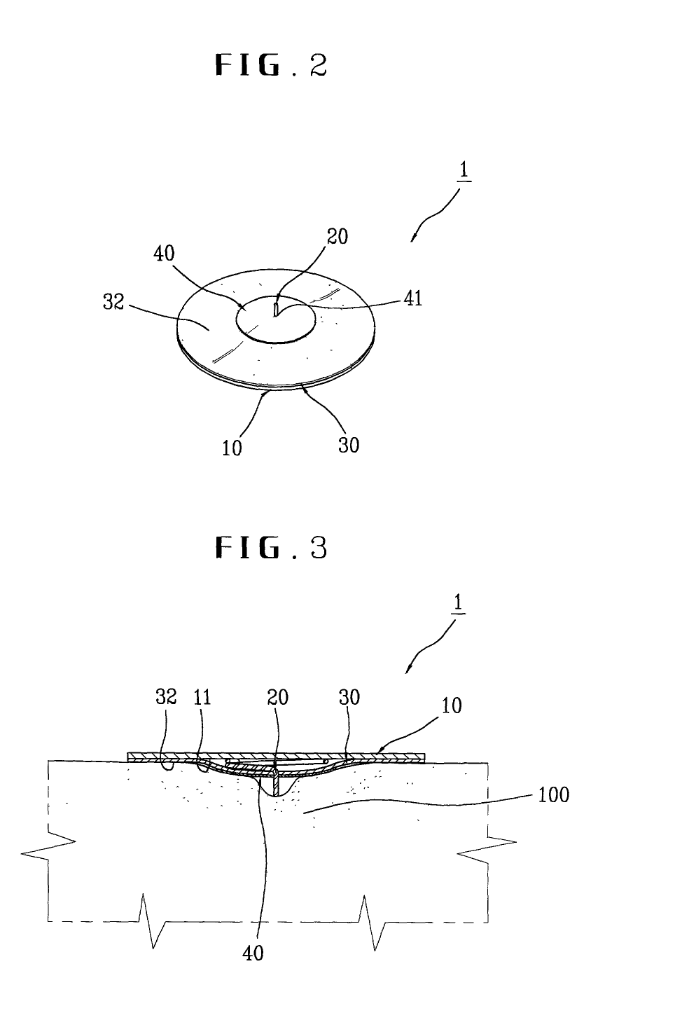

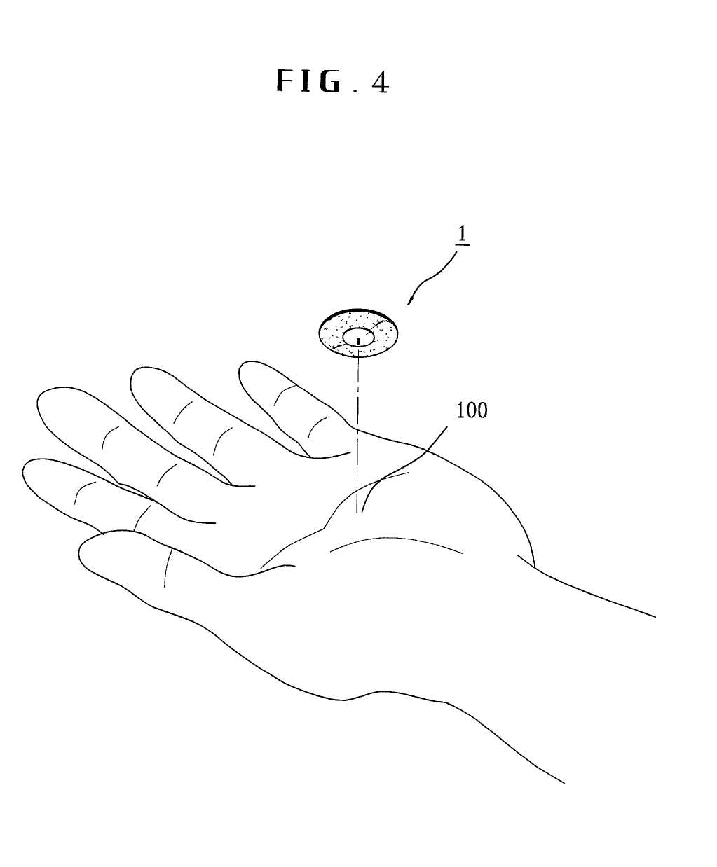

### DESCRIPTION OF THE PREFERRED EMBODIMENTS

A finger pressure device 1 for acupuncture according to the present invention includes an adhesive tape 10 with an adhesive surface 11 on one side; a T-pellet 20 attached on the adhesive surface of said adhesive tape 10; an one-sided tape 30 with a small hole 31 in the center and having an adhesive surface 32 of the same size with said adhesive tape 10; and a gold plate 40 with a small hole in the center through which an end portion of said T-pellet 20 is inserted.

The numeral 100 refers to the skin.

The one-sided tape 30 with the adhesive surface 32 is attached on the T-pellet 20 which is attached on the adhesive surface 11 of the adhesive tape 10. And the gold plate 40 is attached on the adhesive surface 32 of the one-sided tape 30. The T-pellet 20 and the gold plate 40 are combined firmly by the one-sided tape 30 between them.

As shown in FIG. 3, the T-pellet 20 is attached at the Meridian points or the affected parts of the skin 100. The T-pellet 20 is placed at the Meridian points and the gold plate 40 is also placed directly on the skin. The end portion of the T-pellet 20 placed at the Meridian points provides pressure-stimulation effects to the Meridian points and gold of the gold plate 40 has a power of controlling body currents due to its electric transmission.

In traditional chinese medicine, gold is used for medicine pills. For example, invigorating pills are wrapped with thin gold foils and users take the pills without peeling the gold foils. And gold powder is also swallowed by patients because gold is believed to do good for the five viscera and the six entrails and have an effect of stabilizing human spirits.

In case that excessive body currents are transmitted into some parts, the body might get fever, pain, inflammation and so on. The gold plate 40 has an effect of scattering excessive currents. Conversely, in case that some parts of the body get short of required currents, the body might experience psychroalgia, malfunction, paralysis, blood circulation disorder, trembling, spasm and so on. The gold plate 40 has an effect of promoting transmission of body currents.

What is claimed is:

1. A finger pressure device for acupuncture comprising:
an adhesive tape with an adhesive surface on one side;
a T-pellet attached on the adhesive surface of said adhesive tape;
an one-sided tape with a small hole in the center, having an adhesive surface of the same size with said adhesive tape; and
a gold plate with a small hole in the center through which an end portion of said T-pellet is inserted.
